(19) United States
(12) Patent Application Publication
(10) Pub. No.: US 2025/0387610 A1
(43) Pub. Date: Dec. 25, 2025

(54) NEEDLE FREE CONNECTOR PUCK VALVE

(71) Applicant: CAREFUSION 303, INC., San Diego, CA (US)

(72) Inventors: Seyed Alireza SALEHI BOROJERDI, Aliso Viejo, CA (US); Mohammed Mehtab Khan, Karnataka (IN); David Stockey, Holly Springs, NC (US)

(21) Appl. No.: 19/249,921

(22) Filed: Jun. 25, 2025

Related U.S. Application Data
(60) Provisional application No. 63/664,089, filed on Jun. 25, 2024.

(51) Int. Cl. A61M 39/26; A61M 39/24

(52) U.S. Cl. CPC A61M 39/26; A61M 2039/2426; A61M 2039/267; A61M 2205/3331

(57) ABSTRACT

Fluid connectors comprising valves with channels that comprise undulating circumferential ridges are disclosed. A fluid connector includes a housing comprising a post, which is coupled with a valve. The valve is displaceable relative to the post by fluid delivery device, and the displacement allows fluid to flow through the fluid connector assembly from the fluid delivery device. The fluid delivery device applies an external force to the valve when the fluid delivery device is engaged with the fluid connector assembly. When the fluid delivery device is disengaged, the external force is removed, and a resolved force from the post displaces the valve to its original shape and position. In its original shape and position, the valve prevents the fluid from flowing through the fluid connector assembly.

### RELATED APPLICATION DATA

This application claims priority to U.S. Provisional Application No. 63/664,089, filed Jun. 25, 2024, the entire content of which is incorporated herein by reference.

### TECHNICAL FIELD

The present disclosure relates generally to needleless fluid connector systems and, more particularly, to needleless fluid connector systems that incorporate flexible valves.

### BACKGROUND

Medical treatments often include the infusion of a medical fluid (e.g., a saline solution or a liquid medication) to patients using an intravenous (IV) catheter that is connected through an arrangement of flexible tubing and fittings, commonly referred to as an “IV set,” to a source of fluid (e.g., an IV bag). Alternatively, medical fluid can be delivered to patients using syringes.

Both IV sets and syringes can couple to patients through a needleless fluid connector. Needleless fluid connectors incorporate valves that move relative to the center post. In some applications, the valves rely on the viscoelasticity of bellows to regain the valves' original shape and position after the valves have been moved relative to the center post. However, with repeated use, bellows lose viscoelasticity. This loss of viscoelasticity increases the amount of time required for the valve to regain its original shape and position, which, in turn, increases the chance of medical fluid leaking out of the needleless fluid connector.

### SUMMARY

Aspects of the present disclosure provide a needleless connector with a post (e.g., center post) coupled with a valve comprising a channel with undulating circumferential ridges. When engaged with a fluid delivery device (e.g., syringe or IV set), the valve is displaced (e.g., compressed) relative to the post in a downward direction, causing the post to protrude through the valve. As the fluid delivery device is removed from the valve, the interior structure of the valve encourages the valve to return to its original shape and position.

Specifically, as the fluid delivery device is removed from the connector assembly, the force provided by the fluid delivery device on the valve reduces, and the respondent force provided by the post on the valve displaces the valve up the post. The respondent force allows the valve to return to its initial position and cover the opening in the post. When the post no longer protrudes through the valve, the valve prevents upstream flow, leakage, and/or spillage of fluid. Thus, it is important that the valve travels back up the post in a timely and efficient manner.

Furthermore, by exchanging the bellow for the interior structure of the valve, less material is required for the valve. This makes both the valve and the needleless connector \ more compact.

In accordance with at least some embodiments disclosed herein is the realization that valves that rely on bellows to regain shape and position after the valves are displaced relative to the center post become less effective over time. The bellows suffer viscoelastic losses due to repeated compression, which increases the regain time required for the valve to reset. This can result in unintended leakage or spillage of medical fluid.

Accordingly, aspects of the present disclosure provide a fluid connector comprising: a housing comprising a proximal end, a distal end, and an inner chamber having an opening at the proximal end; a post extending from a distal portion of the inner chamber toward the proximal end and comprising an aperture, at a proximal portion of the post, and a lumen fluidly connected to the aperture, the post having a generally conical shape with a diameter at the proximal portion of the post and a larger diameter at the distal portion of the inner chamber; and a valve coupled with the post at the proximal portion of the post and comprising a channel with undulating circumferential ridges, the valve (i) being configured to seal the opening and the aperture in a covered configuration and (ii) is displaced distally by an external force to unseal the opening and the aperture in an uncovered configuration, wherein, when the valve is changed from the covered configuration to the uncovered configuration, the channel elastically and radially expands and generates a resolved force as the channel is expanded and as the valve is displaced distally, wherein, when the external force is removed, the channel is configured to contract radially, and the valve is displaced proximally toward the covered configuration by the resolved force.

Some instances of the present disclosure provide a method for regulating delivery of a medical fluid, the method comprising, by a fluid connector: providing a housing, the housing comprising a proximal end, a distal end, and an inner chamber having an opening at the proximal end; providing a post extending from a distal portion of the inner chamber toward the proximal end and comprising an aperture, at a proximal portion of the post, and a lumen fluidly connected to the aperture; providing a valve comprising a channel with undulating circumferential ridges; and receiving, in the channel, the post; wherein the post has a generally conical shape with a diameter at the proximal portion of the post and a larger diameter at the distal portion of the inner chamber, wherein the valve is configured to seal the opening and the aperture in a covered configuration, wherein the valve is displaced distally by an external force to unseal the opening and the aperture in an uncovered configuration, wherein the fluid connector is configured to form a fluid pathway between the aperture and the lumen in the uncovered configuration, wherein, when the valve is changed from the covered configuration to the uncovered configuration, the channel elastically and radially expands and generates a resolved force as the channel is expanded and as the valve is displaced distally, wherein, when the external force is removed, the channel is configured to contract radially, and the valve is displaced proximally toward the covered configuration by the resolved force.

Accordingly, the present application addresses several operational challenges encountered in prior valves that rely on bellows, including needleless access valves, and provides numerous enhancements and improvements for displacing the valves without suffering viscoelastic losses or Mullins' damage. In the absence of such viscoelastic losses and Mullins' damage, the valve can perform repeatedly (i.e., be displaced and/or compressed repeatedly) over long cycles.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates a fluid connector assembly in use with a fluid delivery device, in accordance with some aspects of the present disclosure.

FIG. 2 illustrates a perspective view of the fluid connector, in accordance with some aspects of the present disclosure.

FIG. 3 illustrates a partial cross-sectional view of the fluid connector assembly, in accordance with some aspects of the present disclosure.

FIGS. 4A and 4B illustrate a front view and a cross-sectional view of a valve of the fluid connector, in accordance with some aspects of the present disclosure.

FIG. 5 illustrates a partial cross-sectional view of the fluid connector before an external force is applied to the valve, in accordance with some aspects of the present disclosure.

FIG. 6 illustrates a partial cross-sectional view of the fluid connector while the external force is applied to the valve, in accordance with some aspects of the present disclosure.

FIG. 7 illustrates a partial cross-sectional view of the fluid connector when the external force is removed from the valve, in accordance with some aspects of the present disclosure.

FIG. 8 illustrates a flowchart showing a method for regulating delivery of a medical fluid by a fluid connector, in accordance with some aspects of the present disclosure.

### DETAILED DESCRIPTION

In the following detailed description, numerous specific details are set forth to provide a full understanding of the subject technology. The subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

Needle-free connectors are essential devices to deliver fluid to a patient via an IV catheter. Needle-free connectors may be used in general patient populations, including neonatal, pediatric, and adult patients. In various applications, the pressure applied to the blood component should not exceed 300 mm Hg (5 psi) as this may result in hemolysis or bag breakage, the IV fluid needs to be injected in bolus without control during power injection and infusion pressure should never exceed 25 psi, as pressure higher than 25 psi may damage blood vessels. Thus, medical professional using needle-free connectors face challenges to maintain the various higher-pressure limits during infusion delivery with typical connectors.

Typical needle-free connectors utilize valves that rely on bellows to regain the original shape and/or length of the valve. However, valves that utilize bellows have drawbacks. For example, the compression of the bellow that is inherent in the use of the needle-free connectors deteriorates the viscoelastic qualities of the bellow. Consequently, over time, the valve becomes less effective in that the valve is slower to return to its original position. This delayed return to the original state of the valve can cause leakage or spillage of medical fluid from the needleless fluid connector. The following devices and methods provide design modifications to overcome the foregoing issues.

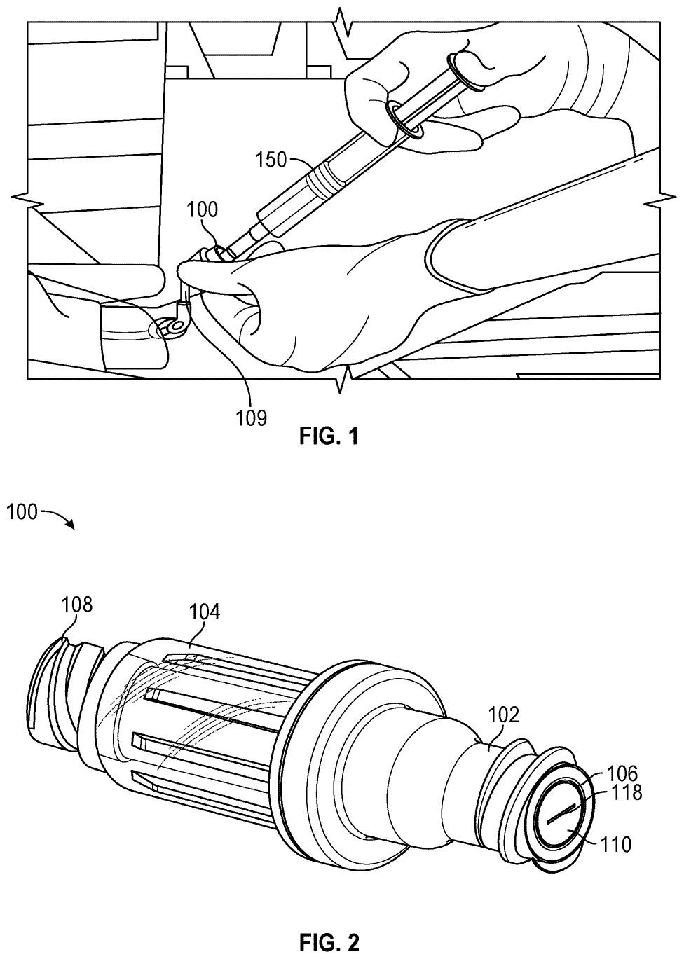

Referring now to the figures, FIG. 1 illustrates a fluid connector 100 in use with a fluid delivery device 150, in accordance with some aspects of the present disclosure. The fluid connector 100 may be used to provide a connection point for a fluid delivery device 150. In some embodiments, the fluid delivery device 150 includes a syringe, such as a needle-free syringe. In some embodiments, the fluid delivery device 150 includes tubing from an IV set. Accordingly, in some embodiments, the connector 100 can take the form of a needle-free connector assembly. For some exemplary IV applications, the fluid delivery device 150 may be used for rapid injections of medication, sometimes referred to as a “push” or “bolus” to quickly send a one-time dose of medication into a patient's bloodstream. The fluid delivery device 150 is connected to the opening 106 (shown in FIG. 2) of the connector 100. Additionally, the fluid outlet 108 of the connector 100 can be connected to a catheter line. The valve 110 is positioned to regulate the flow of fluid provided by the fluid delivery device 150 to the catheter line.

FIG. 2 illustrates a perspective view of the fluid connector 100, in accordance with some aspects of the present disclosure. The connector assembly 100 includes a housing 102. The housing 102 includes a proximal end, a distal end, an inner chamber having an opening 106 at the proximal end, and a fluid outlet 108. As shown, the housing 102, the opening 106, and the fluid outlet 108 are cylindrical, or generally cylindrical, bodies with circular cross sections. Other shapes are also possible.

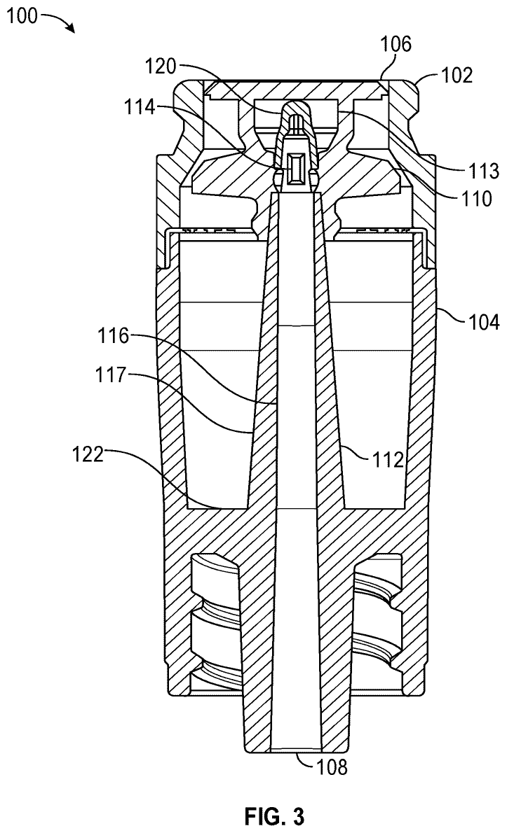

FIG. 3 illustrates a partial cross-sectional view of the fluid connector assembly 100, in accordance with some aspects of the present disclosure. The housing 102 may include a post 112 (i.e., a center post) that extends from a distal portion 122 of the inner chamber toward the proximal end of the housing 102. The post 112 has a generally conical shape, such that the post 112 has a diameter at the proximal portion 120 of the post and a larger diameter at the distal portion 122 of the inner chamber. The post 112 includes a lumen 116 that is fluidly connected to the aperture 114 to facilitate the transfer of fluid through the connector assembly 100. The post 112 also includes an aperture 114 (representative of one or more openings in the post 112) near the proximal portion 120 of the post 112. For purposes of illustration, the post 112 in FIG. 3 is rotated such that the aperture 114 is viewable. The aperture 114 of the post 112 is designed to receive fluid from the opening 106 of the housing 102. Accordingly, the opening 106 can be fluidly connected to the aperture 114 if the aperture 114 is exposed. The opening 106 of the housing 102 includes a size and shape that allows the fluid delivery device 150 to enter the housing 102 through the opening 106 and expose the aperture 114 by displacing the valve 110.

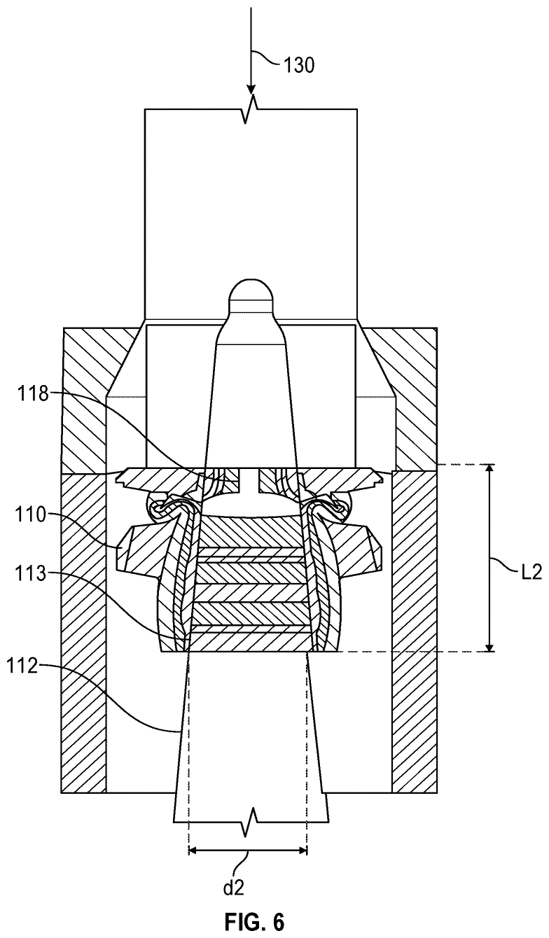

The valve 110 includes a channel 113 that receives the post 112. The valve 110 surrounds the post 112. In some embodiments, the valve 110 is disposed on, and engaged with, the post 112. Based on the position of the valve 110 shown in FIG. 3, the valve 110 is in a covered position and covers the aperture 114, thereby preventing the flow of fluid into the aperture 114 and to the lumen 116. However, the valve 110 is designed to regulate flow based in part upon the displacement (e.g., compression) of the valve 110 by an external force 130 (shown in FIG. 6) such that the aperture 114 is uncovered by the valve 110, as will be shown and described in detail below. The external force 130 may be applied by a fluid delivery device 150. It should be noted that displacement of the valve 110 may include an elastic displacement (e.g., elastic compression), thus allowing the valve 110 to return, after displacement, to its original form (shown in FIG. 3) when a fluid delivery device 150 is removed/disengaged from the valve 110.

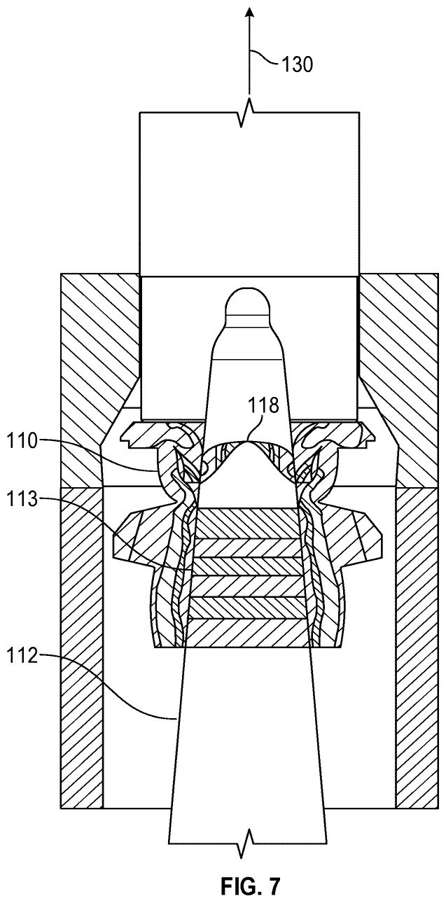

Additionally, a slit 118 (shown in FIGS. 6 and 7), representing a cut or other discontinuity in the valve 110, is formed in the valve 110. In the covered position of the valve 110, no object(s) is/are positioned in the slit 118 of the valve 110 and the slit 118 is generally closed. For example, a proximal portion 120 of the post 112 does not protrude through the slit 118 in the covered position of the valve 110. When the slit 118 is closed, fluid will not flow through the valve 110 into the aperture 114 of the post 112 and to the lumen 116.

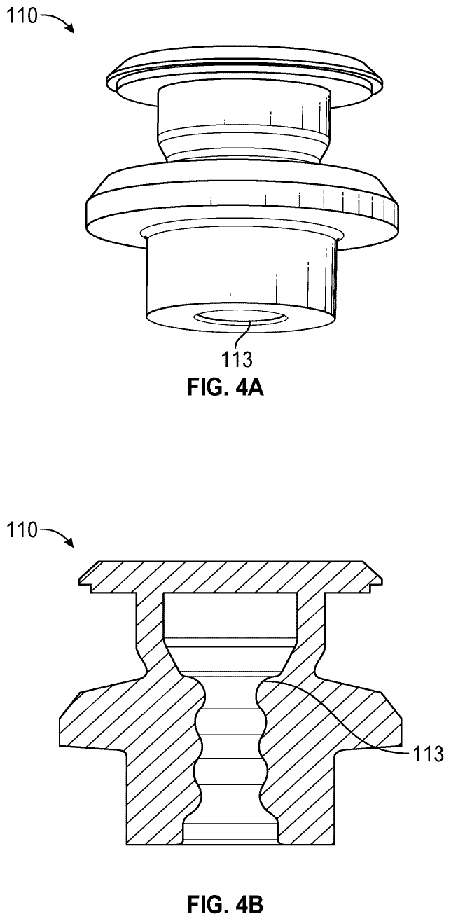

FIGS. 4A and 4B illustrate a front view and a cross-sectional view of a valve 110 of the fluid connector 100, in accordance with some aspects of the present disclosure. The valve 110 comprises intrusions and protrusions such that the valve 110 has contact surfaces that align with different portions of the housing 102. The interior of the valve 110 comprises a channel 113. In some embodiments, the channel 113 has a cylindrical, or generally cylindrical, body with circular cross sections. The diameter of the channel 113 changes from the bottom of the valve 110 to the top of the valve 110. In other words, the channel 113 comprises circumferential undulating ridges. The grooves and/or intruding portions between the ridges maintain weaker contact with the post 112 than the protruding portions of the ridges. In some embodiments, the grooves between the ridges have no contact with the post 112. Consequently, this structure limits friction between the valve 110 and the post 112.

The present disclosure utilizes the undulating circumferential ridges in the channel of the valve 110 in place of a bellow. Unlike a bellow, which would suffer viscoelastic losses and Mullin's damage, the undulating circumferential ridges will prevent the valve from acquiring an increased regaining time (i.e., taking longer to transport from the open position 110 to the closed position). Avoiding an increased regaining time also decreases the chances of medical fluid leaking or spilling out of the post 112 by enabling the valve 110 to quickly recover or reconceal the aperture 114.

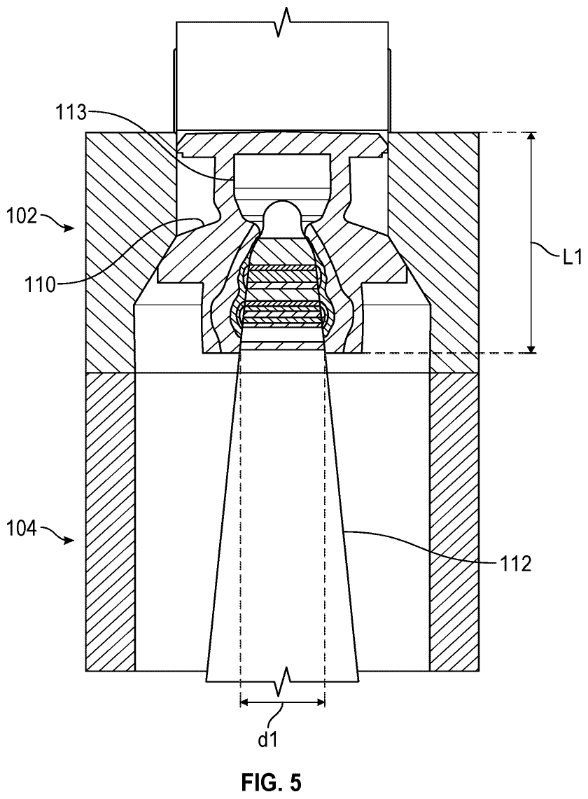

FIG. 5 illustrates a partial cross-sectional view of the fluid connector 100 before an external force 130 is applied to the valve 110, in accordance with some aspects of the present disclosure. Before the external force 130 is applied by the fluid delivery device 150, the fluid connector 100 is in the covered position. The valve 110 has not been pushed or compressed down the post 112 such that the aperture 114 is exposed. Consequently, the opening 106 is not yet fluidly connected to the aperture 114 and lumen 116. Additionally, before the external force 130 is applied to the valve 110, the valve 110 has a length L1, and the channel 113 has a diameter d1.

FIG. 6 illustrates a partial cross-sectional view of the fluid connector 100 while the external force 130 is applied to the valve 110, in accordance with some aspects of the present disclosure. Based on the displacement of the valve 110 by the external force 130, the valve 110 is in the uncovered position and can receive fluid from the fluid delivery device 150. In the uncovered position of the valve 110, the proximal portion 120 of the post 112 protrudes through the slit 118 of the valve 110. Additionally, the displacement of the valve 110 exposes the aperture 114 of the post 112. When the aperture 114 is uncovered by the valve 110, fluid flows from the fluid delivery device 150 through the slit 118 (now open) and subsequently to the lumen 116 by way of the aperture 114. Accordingly, the slit 118 is fluidly connected to the aperture 114, the lumen 116, and the fluid outlet 108.

The valve 110 compresses or reduces to a length L2 (less than the length L1 shown in FIG. 5) based on the external force 130 applied by the fluid delivery device 150. When the valve 110 is compressed by the external force 130, the channel 113 has a diameter d2 (larger than the diameter d1 shown in FIG. 5). The compression of the valve 110 represents relative movement of the valve 110 as compared to the housing 102 and the post 112.

As the external force 130 displaces the valve 110 from the proximal portion 120 of the post 112 to the distal portion 122 of the inner chamber, the channel 113 elastically and radially expands because the diameter of the post 112 is increasing. The channel 113 squeezes the post 112 more tightly as the valve 110 is displaced from the narrower end of the post 112 (with diameter d1) to the wider end of the post 112 (with diameter d2). In response to the increasing force from the valve 110, the post 112 exerts a resolved force on the valve 110.

FIG. 7 illustrates a partial cross-sectional view of the fluid connector 100 when the external force 130 is removed from the valve 110, in accordance with some aspects of the present disclosure. As the external force 130 is removed from the valve 110, the channel 113 begins to contract radially, and the resolved force displaces the valve 110 proximally (i.e., back into the covered configuration). In the covered configuration, the valve 110 covers the aperture 114. As a result, the valve 110 prevents fluid (external to the valve 110) from subsequently entering the lumen 116.

In some embodiments, the valve 110 is still in the uncovered position because the valve 110 has not necessarily slid far enough up the post 112 to cover the aperture 114. In some embodiments, the valve 110 may cover the aperture 114 prior to the fluid delivery device 150 being fully removed from the opening 106 of the housing 102 and prior to the fluid delivery device 150 being fully disengaged from the valve 110. When the fluid delivery device 150 is removed from the opening 106 of the housing 102 and no longer engages the valve 110, the valve 110 is no longer displaced and can return to its original shape (with length L1 and the channel 113 having a diameter d1) and position with respect to the post 112. At this point, the valve 110 is in the covered position and covers the aperture 114.

Further, the undulating circumferential ridges of the channel 113 of the valve 110 limit the amount of friction between the valve 110 and the post 112. The limited friction helps the valve 110 transition from length L2 back to length L1 in an efficient manner. That is, the limited friction enables the valve 110 to return quickly to its original shape and position and prevents the valve 110 from getting stuck in the compressed, open position. This reduces the chances of leakage and/or spillage of fluid through the aperture 114 of the post 112.

The undulating circumferential ridges of the channel 113 also influence the magnitude of the resolved force such that the resolved force is sufficient to displace the valve 110 proximally back into the covered configuration when the external force 130 is removed.

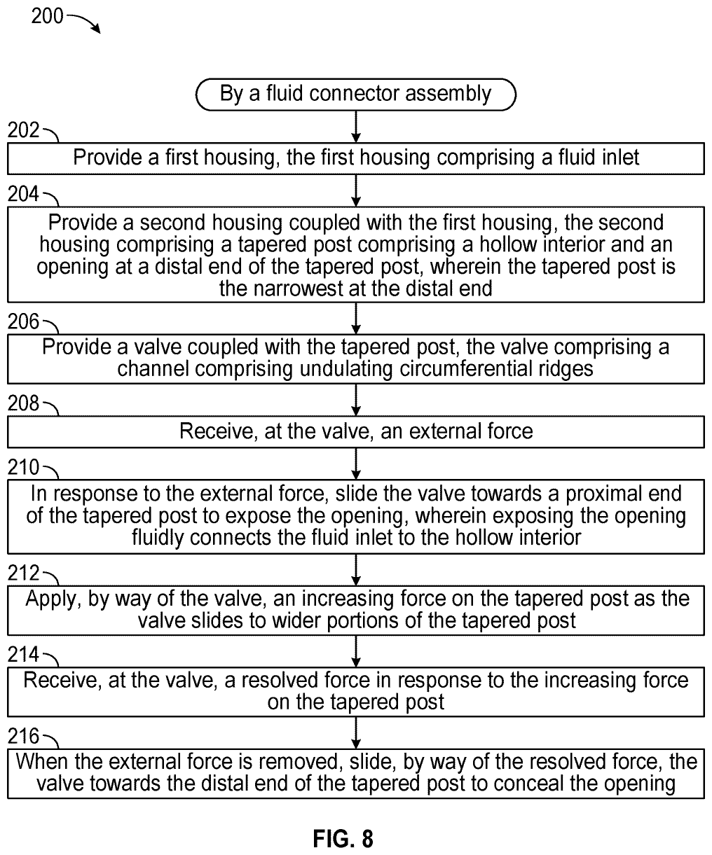

FIG. 8 illustrates a flowchart showing a method for regulating delivery of a medical fluid by a fluid connector, in accordance with some aspects of the present disclosure. The method shown in the flowchart 200 may be performed by fluid connectors described herein. Accordingly, fluid connectors described herein can carry out the method shown in the flowchart 200.

In step 202, a housing is provided. The first housing comprises a proximal end, a distal end, and an inner chamber having an opening at the proximal end.

In step 204, a post is provided. The post extends from a distal portion of the inner chamber toward the proximal end and comprises an aperture and a lumen fluidly connected to the aperture. The aperture is positioned at a proximal portion of the post. The post has a generally conical shape with a diameter at the proximal portion of the post and a larger diameter at the distal portion of the inner chamber.

In step 206, a valve is provided. The valve is coupled with the post and comprises a channel comprising undulating circumferential ridges. The undulating circumferential ridges restrict friction between the channel and the post by limiting surface contact between the channel and the post. The valve is configured to seal the opening and the aperture in a covered configuration. The valve is displaced distally by an external force to unseal the opening and the aperture in an uncovered configuration. The fluid connector is configured to form a fluid pathway between the aperture and the lumen in the uncovered configuration. When the valve is changed from the covered configuration to the uncovered configuration, the channel elastically and radially expands and generates a resolved force as the channel is expanded and as the valve is displaced distally. Similarly, when the external force is removed, the channel is configured to contract radially, and the valve is displaced proximally toward the covered configuration by the resolved force.

In some embodiments, the undulating circumferential ridges restrict friction between the channel and the post, which encourages the channel to contract radially and the valve to be displaced proximally toward the covered configuration by the resolved force when the external force is removed.

In some embodiments, the valve comprises a first length before the external force is applied, and the valve comprises a second length when the external force is applied, the second length being shorter than the first length. Likewise, the channel comprises a first diameter before the external force is applied, and the valve comprises a second diameter when the external force is applied, the second diameter being larger than the first diameter.

In some embodiments, the external force is applied by a fluid delivery device.

In some embodiments, displacing the valve towards a portion of the post with the larger diameter comprises penetrating a slit of the valve with the post.

In step 208, the post is received in the channel.

Although the present disclosure includes embodiments in which a post includes a single opening in the Figures, it should be understood that the post may include any number of openings, each of each can receive a fluid from a fluid delivery device.

The features of the present disclosure provide multiple components (e.g., a fluid delivery device and a fluid connector) can be coupled together to form a fluid pathway therebetween. When coupled together, the features of the present disclosure resist unintentional separation between the components. However, if the components are separated, wither unintentionally or intentionally, the fluid pathway through the components may become closed or obstructed to prevent fluid loss therefrom. The features of the present disclosure as provided that upon separation of the components, any of the components can be cleaned and disinfected, and the components can be once again coupled together to form a fluid pathway therebetween.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 9, or clause 16. The other clauses can be presented in a similar manner.

Clause 1. A fluid connector comprising: a housing comprising a proximal end, a distal end, and an inner chamber having an opening at the proximal end; a post extending from a distal portion of the inner chamber toward the proximal end and comprising an aperture, at a proximal portion of the post, and a lumen fluidly connected to the aperture, the post having a generally conical shape with a diameter at the proximal portion of the post and a larger diameter at the distal portion of the inner chamber; and a valve coupled with the post at the proximal portion of the post and comprising a channel with undulating circumferential ridges, the valve (i) being configured to seal the opening and the aperture in a covered configuration and (ii) is displaced distally by an external force to unseal the opening and the aperture in an uncovered configuration, wherein, when the valve is changed from the covered configuration to the uncovered configuration, the channel elastically and radially expands and generates a resolved force as the channel is expanded and as the valve is displaced distally, wherein, when the external force is removed, the channel is configured to contract radially, and the valve is displaced proximally toward the covered configuration by the resolved force.

Clause 2. The fluid connector assembly of clause 0, wherein the undulating circumferential ridges are configured to restrict friction between the channel and the post by limiting surface contact between the channel and the post, which encourages the channel to contract radially and the valve to be displaced proximally toward the covered configuration by the resolved force when the external force is removed.

Clause 3. The fluid connector assembly of clause 0, wherein the valve extends circumferentially around the post.

Clause 4. The fluid connector assembly of clause 0, wherein the valve comprises a first length before the external force is applied, and the valve comprises a second length when the external force is applied, the second length being shorter than the first length.

Clause 5. The fluid connector assembly of clause 0, wherein the valve comprises a slit, and the opening is exposed when the post protrudes through the slit.

Clause 6. The fluid connector assembly of clause 0, wherein the post is a cannula.

Clause 7. The fluid connector assembly of clause 0, wherein the external force is applied by a fluid delivery device.

Clause 8. A method for regulating delivery of a medical fluid, the method comprising, by a fluid connector: providing a housing, the housing comprising a proximal end, a distal end, and an inner chamber having an opening at the proximal end; providing a post extending from a distal portion of the inner chamber toward the proximal end and comprising an aperture, at a proximal portion of the post, and a lumen fluidly connected to the aperture; providing a valve comprising a channel with undulating circumferential ridges; and receiving, in the channel, the post; wherein the post has a generally conical shape with a diameter at the proximal portion of the post and a larger diameter at the distal portion of the inner chamber, wherein the valve is configured to seal the opening and the aperture in a covered configuration, wherein the valve is displaced distally by an external force to unseal the opening and the aperture in an uncovered configuration, wherein the fluid connector is configured to form a fluid pathway between the aperture and the lumen in the uncovered configuration, wherein, when the valve is changed from the covered configuration to the uncovered configuration, the channel elastically and radially expands and generates a resolved force as the channel is expanded and as the valve is displaced distally, wherein, when the external force is removed, the channel is configured to contract radially, and the valve is displaced proximally toward the covered configuration by the resolved force.

Clause 9. The method of claim 8, wherein the undulating circumferential ridges are configured to restrict friction between the channel and the post by limiting surface contact between the channel and the post, which enables the channel to contract radially and the valve to be displaced proximally toward the covered configuration by the resolved force when the external force is removed.

Clause 10. The method of clause 8, wherein the external force is applied by a fluid delivery device.

Clause 11. The method of clause 8, wherein displacing the valve towards a portion of the post with the larger diameter comprises penetrating a slit of the valve with the post.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear,” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A fluid connector comprising:
a housing comprising a proximal end, a distal end, and an inner chamber having an opening at the proximal end;
a post extending from a distal portion of the inner chamber toward the proximal end and comprising an aperture, at a proximal portion of the post, and a lumen fluidly connected to the aperture, the post having a generally conical shape with a diameter at the proximal portion of the post and a larger diameter at the distal portion of the inner chamber; and
a valve coupled with the post at the proximal portion of the post and comprising a channel with undulating circumferential ridges, the valve (i) being configured to seal the opening and the aperture in a covered configuration and (ii) is displaced distally by an external force to unseal the opening and the aperture in an uncovered configuration,
wherein, when the valve is changed from the covered configuration to the uncovered configuration, the channel elastically and radially expands and generates a resolved force as the channel is expanded and as the valve is displaced distally,
wherein, when the external force is removed, the channel is configured to contract radially, and the valve is displaced proximally toward the covered configuration by the resolved force.

2. The fluid connector of claim 1, wherein the undulating circumferential ridges are configured to restrict friction between the channel and the post by limiting surface contact between the channel and the post, which encourages the channel to contract radially and the valve to be displaced proximally toward the covered configuration by the resolved force when the external force is removed.

3. The fluid connector of claim 1, wherein the valve extends circumferentially around the post.

4. The fluid connector of claim 1, wherein the valve comprises a first length before the external force is applied, and the valve comprises a second length when the external force is applied, the second length being shorter than the first length.

5. The fluid connector of claim 1, wherein the valve comprises a slit, and the opening is exposed when the post protrudes through the slit.

6. The fluid connector of claim 1, wherein the post is a cannula.

7. The fluid connector of claim 1, wherein the external force is applied by a fluid delivery device.

8. A method for regulating delivery of a medical fluid, the method comprising, by a fluid connector:
providing a housing, the housing comprising a proximal end, a distal end, and an inner chamber having an opening at the proximal end;
providing a post extending from a distal portion of the inner chamber toward the proximal end and comprising an aperture, at a proximal portion of the post, and a lumen fluidly connected to the aperture;
providing a valve comprising a channel with undulating circumferential ridges; and
receiving, in the channel, the post;
wherein the post has a generally conical shape with a diameter at the proximal portion of the post and a larger diameter at the distal portion of the inner chamber,
wherein the valve is configured to seal the opening and the aperture in a covered configuration,
wherein the valve is displaced distally by an external force to unseal the opening and the aperture in an uncovered configuration,
wherein the fluid connector is configured to form a fluid pathway between the aperture and the lumen in the uncovered configuration,
wherein, when the valve is changed from the covered configuration to the uncovered configuration, the channel elastically and radially expands and generates a resolved force as the channel is expanded and as the valve is displaced distally,
wherein, when the external force is removed, the channel is configured to contract radially, and the valve is displaced proximally toward the covered configuration by the resolved force.

9. The method of claim 8, wherein the undulating circumferential ridges are configured to restrict friction between the channel and the post by limiting surface contact between the channel and the post, which enables the channel to contract radially and the valve to be displaced proximally toward the covered configuration by the resolved force when the external force is removed.

10. The method of claim 8, wherein the external force is applied by a fluid delivery device.

11. The method of claim 8, wherein displacing the valve towards a portion of the post with the larger diameter comprises penetrating a slit of the valve with the post.

12. A fluid connector comprising:
a housing comprising a proximal end, a distal end, and an inner chamber having an opening at the proximal end;
a post extending toward the proximal end and comprising an aperture and a lumen fluidly connected to the aperture, the post having a generally conical shape with a diameter at a proximal portion of the post and a larger diameter at a distal portion of the inner chamber; and
a valve coupled with the post at the proximal portion of the post and comprising a channel with undulating circumferential ridges, the valve (i) being configured to seal the opening and the aperture in a covered configuration and (ii) is displaced distally by an external force to unseal the opening and the aperture in an uncovered configuration.

13. The fluid connector of claim 12, wherein, when the valve is displaced distally, the channel elastically and radially expands and generates a proximally directed force that urges the valve proximally along the post.

14. The fluid connector of claim 13, wherein the valve channel is configured to contract radially such that the valve displaces proximally toward the covered configuration when the external force is removed.

15. The fluid connector of claim 12, wherein the undulating circumferential ridges are configured to limit friction between the channel and the post by limiting surface contact between the channel and the post.

16. The fluid connector of claim 12, wherein the valve channel extends circumferentially around the post.

17. The fluid connector of claim 12, wherein the valve is configured to compressed axially when the external force is applied.

18. The fluid connector of claim 12, wherein the valve comprises a slit, and the opening is exposed when the post protrudes through the slit.

19. The fluid connector of claim 1, wherein the valve is configured to displace distally by a fluid delivery device.
